(19) United States
(12) Patent Application Publication
(10) Pub. No.: US 2025/0143598 A1
(43) Pub. Date: May 8, 2025

(54) RECEPTION COIL UNIT AND MEDICAL IMAGE DIAGNOSIS SYSTEM

(71) Applicant: FUJIFILM Corporation, Tokyo (JP)

(72) Inventors: Hisaaki OCHI, Tokyo (JP); Yosuke OTAKE, Tokyo (JP); Masayoshi DOHATA, Tokyo (JP)

(73) Assignee: FUJIFILM Corporation, Tokyo (JP)

(21) Appl. No.: 18/937,937

(22) Filed: Nov. 5, 2024

(30) Foreign Application Priority Data
Nov. 7, 2023 (JP) 2023-190145

(51) Int. Cl. A61B 5/055; G01R 33/34; G01R 33/36

(52) U.S. Cl. CPC A61B 5/055; G01R 33/34084; G01R 33/3642

(57) ABSTRACT

Provided are a receive coil unit and a medical image diagnosis system capable of reducing a burden on imaging staff. A receive coil unit includes: a receive coil of which one end side of a table, on which a subject is placed, is attached to one side portion among both side portions of the table, that has one sheet accommodating a plurality of sub coil channels, and that has a member to be locked provided on a surface of the sheet; and a fixing member that is attached to the other side portion of the table, that has a locking member locked to the member to be locked, and that fixes the receive coil to be length-adjustable by adjusting a locking position of the member to be locked with respect to the locking member.

### CROSS-REFERENCE TO RELATED APPLICATION

The present application claims priority under 35 U.S.C § 119(a) to Japanese Patent Application No. 2023-190145 filed on Nov. 7, 2023, which is hereby expressly incorporated by reference, in its entirety, into the present application.

### BACKGROUND OF THE INVENTION

### 1. Field of the Invention

The present invention relates to a receive coil unit and a medical image diagnosis system, and particularly, to a technique for reducing a burden on imaging staff involved in a magnetic resonance imaging apparatus.

### 2. Description of the Related Art

In a case where a subject is imaged by a magnetic resonance imaging (MRI) apparatus, the subject is disposed in a bore of a gantry together with a table of a bed device. In this case, the subject is placed on a top plate of the table, and a receive coil such as a radio frequency (RF) coil is mounted on an imaging part (for example, an abdomen).

In this case, JP2019-92935A discloses a technique of separately using a fixing belt in order to fix a receive coil to a subject. According to JP2019-92935A, two work steps, a work of mounting a receive coil on an imaging part and a work of fixing the receive coil to a table together with a subject by a fixing belt are required.

On the other hand, JP6005279B discloses a configuration in which the two work steps described above are realized by one work step by integrating a receive coil and a fixing belt by a connector connection.

In recent years, in order to save the labor of an imaging staff for selecting a receive coil having a size corresponding to a physique and an imaging part of a subject from among a plurality of receive coils, a sheet-shaped receive coil having a wide area and being deformable may be used.

JP2020-124345A discloses, as one aspect, a foldable receive coil, such as a folding screen, having excellent storability and portability.

In addition, JP2012-130701A discloses a blanket-shaped receive coil fixed to both left and right side portions of a top plate by a fastener as another form.

### SUMMARY OF THE INVENTION

In JP2019-92935A, since the receive coil and the fixing belt are originally separate from each other, there is a problem that the above two work steps are required to fix the receive coil and the subject to the top plate. That is, there is a problem that the burden on the imaging staff is increased.

On the other hand, since JP6005279B has a configuration in which two receive coil units disposed on both left and right side portions of a table are mounted in an overlapping manner on a subject, there may be a case in which the configuration cannot be applied depending on the physique of the subject.

In addition, JP2020-124345A does not disclose a configuration for fixing the receive coil to the table, and JP2012-130701A discloses only a configuration for fixing a blanket-shaped coil having a predetermined size to the table using left and right fasteners. That is, a configuration for reducing a burden on the imaging staff is not disclosed.

The present invention has been made in view of such circumstances, and an object thereof is to provide a receive coil unit and a medical image diagnosis system capable of reducing a burden on imaging staff.

A receive coil unit according to a first aspect of the present invention comprises: a receive coil of which one end side of a table, on which a subject is placed, is attached to one side portion among both side portions of the table, that has one sheet accommodating a plurality of sub coil channels, and that has a member to be locked provided on a surface of the sheet; and a fixing member that is attached to the other side portion opposite to the one side portion of the table, that has a locking member locked to the member to be locked, and that fixes the receive coil to be length-adjustable by adjusting a locking position of the member to be locked with respect to the locking member.

According to the receive coil unit according to a first aspect of the present invention, since the subject can be fixed to the table in one work step of locking the member to be locked of the receive coil to the locking member of the fixing member, it is possible to reduce the burden on the imaging staff. In addition, since the length of the receive coil can be adjusted by changing the locking position of the member to be locked with respect to the locking member, the present invention can be applied regardless of the physique of the subject.

In a receive coil unit according to a second aspect of the present invention, in the first aspect, it is preferable that the member to be locked and the locking member are surface fasteners.

In a receive coil unit according to a third aspect of the present invention, in the second aspect, it is preferable that the member to be locked is provided on a surface opposite to a surface on a subject side among two front and back surfaces of the sheet.

In a receive coil unit according to a fourth aspect of the present invention, in any one of the first to third aspects, it is preferable that the fixing member has an escape hole for leading an extra length portion of the receive coil after length adjustment to an outside of the table.

In a receive coil unit according to a fifth aspect of the present invention, in any one of the first to fourth aspects, it is preferable that the receive coil is foldable.

In the receive coil unit according to a sixth aspect of the present invention, in any one of the first to fifth aspects, it is preferable that the receive coil has gradations indicating a length of the sheet, and the fixing member has an indicator indicating a gradation corresponding to the locking position of the member to be locked with respect to the locking member among the gradations.

A medical image diagnosis system according to a seventh aspect of the present invention comprises: the receive coil unit according to any one of the first to sixth aspects; and a magnetic resonance imaging apparatus.

In a medical image diagnosis system according to an eighth aspect of the present invention, in the seventh aspect, it is preferable that the receive coil has gradations indicating a length of the sheet, the fixing member has an indicator indicating a gradation corresponding to the locking position of the member to be locked with respect to the locking member among the gradations, and the magnetic resonance imaging apparatus includes a reading unit configured to read the gradation indicated by the indicator, and a selection unit configured to select a sub coil channel to be driven among the plurality of sub coil channels corresponding to the gradation read by the reading unit.

In a medical image diagnosis system according to a ninth aspect of the present invention, in the seventh aspect, it is preferable that the receive coil has gradations indicating a length of the sheet, the fixing member has an indicator indicating a gradation corresponding to the locking position of the member to be locked with respect to the locking member among the gradations, and the magnetic resonance imaging apparatus includes an input unit configured to input the gradation indicated by the indicator, and a selection unit configured to select a sub coil channel to be driven among the plurality of sub coil channels corresponding to the gradation input by the input unit.

According to the present invention, it is possible to reduce the burden on the imaging staff.

### BRIEF DESCRIPTION OF THE DRAWINGS

FIG. 1 is an external perspective view of a medical diagnosis system according to an embodiment.

FIG. 2 is a schematic diagram showing an internal configuration of an MRI apparatus.

FIG. 3 is an explanatory diagram showing a configuration of a main part of a receive coil.

FIGS. 4A and 4B are perspective views showing a state where a subject is fixed to a table by a receive coil unit.

FIG. 5 is an explanatory diagram showing a main part of the receive coil unit comprising the receive coil provided with gradations and a fixing member provided with an indicator.

FIGS. 6A and 6B are explanatory diagrams showing a sub coil channel controlled in ON/OFF control.

FIG. 7 is a main part perspective view showing a first modification example.

FIG. 8 is an explanatory diagram showing a second modification example.

FIGS. 9A and 9B are perspective views showing a state where the subject is fixed to the table according to a third modification example.

FIG. 10 is an explanatory diagram showing a form of a slide type receive coil unit.

FIGS. 11A and 11B are explanatory diagrams showing two slide positions of the receive coil unit.

### DESCRIPTION OF THE PREFERRED EMBODIMENTS

Hereinafter, embodiments of a receive coil unit and a medical image diagnosis system according to an embodiment of the present invention will be described with reference to the accompanying drawings.

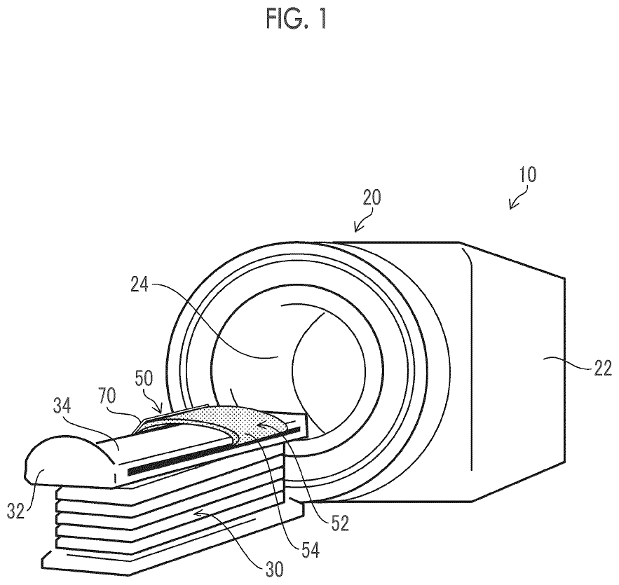

FIG. 1 is an external perspective view of a medical image diagnosis system 10 according to the embodiment of the present invention.

As illustrated in FIG. 1, a medical image diagnosis system 10 according to the embodiment comprises a magnetic resonance imaging apparatus (hereinafter, referred to as an MRI apparatus) 20 and a receive coil unit 50. The MRI apparatus 20 and the receive coil unit 50 are examples of a magnetic resonance imaging apparatus and a receive coil unit according to the embodiment of the present invention.

The MRI apparatus 20 is installed in an examination room of an image diagnosis facility. In the examination room, the subject is placed on a top plate 34 of a table 32 of a bed device 30, and then is transported toward a gantry 22 of the MRI apparatus 20 by a movement operation of the table 32.

The MRI apparatus 20 includes a gantry 22. The gantry 22 has a bore 24 that is an imaging space on a cylinder, and the table 32 moves in the bore 24.

### Internal Configuration of MRI Apparatus

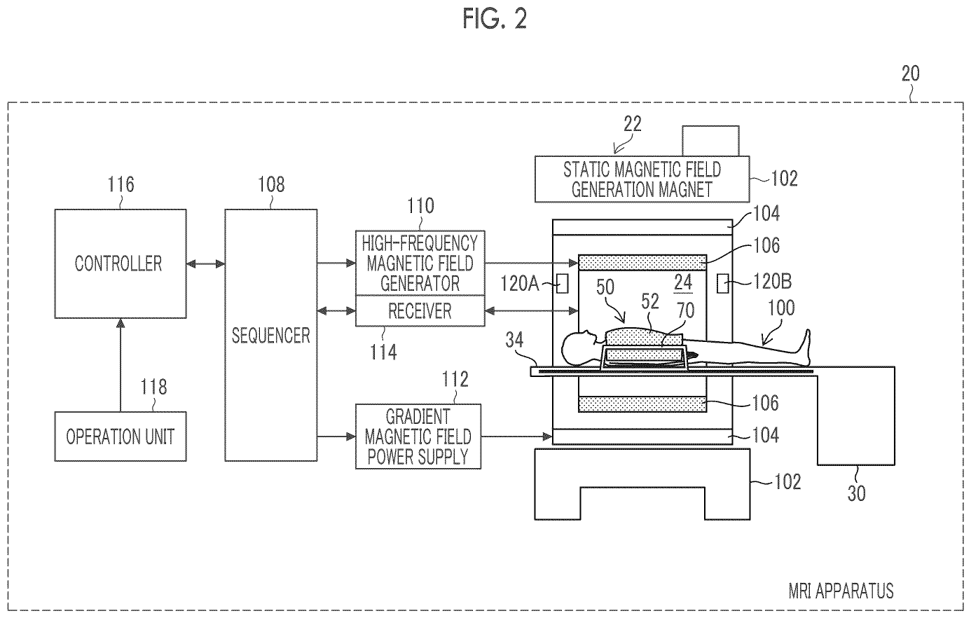

FIG. 2 is a schematic diagram showing an internal configuration of the MRI apparatus 20.

As shown in FIGS. 1 and 2, the MRI apparatus 20 according to the present example comprises a static magnetic field generation magnet 102, a gradient magnetic field coil 104, a radio frequency (RF) coil (hereinafter, referred to as a transmitting coil) 106, and a receive coil 52.

The static magnetic field generation magnet 102 generates a uniform static magnetic field in the bore 14 in which the subject 100 is disposed. The gradient magnetic field coil 104 generates a gradient magnetic field in the bore 14. The transmitting coil 106 generates a high-frequency magnetic field for causing a nucleus magnetic resonance (NMR) signal (hereinafter, referred to as an NMR signal) in an atomic nucleus constituting a tissue of the subject 100. The receive coil 52 detects the NMR signal generated from the subject 100.

The subject 100 placed on the top plate 34 of the table 32 is moved to the bore 24 by moving the table 32, so that the examination site of the subject 100 is positioned at the center of the static magnetic field of the bore 24.

The sequencer 108 sends commands to the high-frequency magnetic field generator 110 and the gradient magnetic field power supply 112 in accordance with an imaging sequence (pulse sequence) to generate a high-frequency magnetic field and a gradient magnetic field, respectively.

The generated high-frequency magnetic field is applied to the subject 100 as a pulsed high-frequency magnetic field (RF pulse) via the transmitting coil 106. The NMR signal generated from the subject 100 is received by the receive coil 52, and the demodulation is performed by the receiver 114.

The gradient magnetic field coil 104 is configured with three gradient magnetic field coils in the three directions, X, Y, and Z directions, respectively, and generates a gradient magnetic field in accordance with a signal from the gradient magnetic field power supply 112.

A nuclear magnetic resonance frequency (demodulation reference frequency f0) to be used as a reference for demodulation in the receiver 114 is set by the sequencer 108. The sequencer 108 controls each unit to operate at a timing and intensity programmed in advance. Among programs, a program that particularly describes the timing and intensity of RF pulses, gradient magnetic fields, and signal reception is referred to as a pulse sequence.

Various pulse sequences depending on the purpose are known, but the detailed description thereof will be omitted here.

A controller 116 controls an operation of the MRI apparatus 20 via the sequencer 108, and receives the signal detected by the receiver 114 and performs various types of signal processing, such as image reconstruction. The receiver 114 quadrature-detects the echo signal (NMR signal) that is an analog wave having the set demodulation reference frequency f0, converts the echo signal into raw data, and then transmits the raw data to the controller 116. This raw data is also referred to as an echo signal or measurement data.

The controller 116 receives various instruction inputs from the operation unit 118 to integrally control each unit of the MRI apparatus 20. Further, the controller 116 performs processing of performing inverse Fourier transformation on the echo signal in the spatial frequency domain received through the sequencer 108 to convert the echo signal into an image in a real space, and generates an MRI image.

The controller 116 is realized by a general-purpose computer, such as a personal computer or a microcomputer. The controller 116 comprises a central processing unit (CPU), a read only memory (ROM), a random access memory (RAM), an input/output interface, and the like.

In the controller 116, various programs, such as the control program stored in the ROM, are developed in the RAM, and the program developed in the RAM is executed by the CPU. Accordingly, the functions of the each unit of the MRI apparatus 20 are realized, and various types of operation processing and control processing are executed via the input/output interface.

In addition, the controller 116 receives the video input from the cameras 120A and 120B described below or the instruction input from the operation unit 118 and performs the control of driving only the sub coil channels necessary for imaging among the plurality of sub coil channels constituting the receive coil 52. This will be described below.

The operation unit 118 includes a mouse, a keyboard, and the like, and functions as a part of a graphical user interface (GUI) that receives an input from an imaging staff by using a display operation window of a display (not shown). The operation unit 118 may be a touch panel type operation unit or may be a tablet type device in which a display function of an image and a menu and a touch panel input function are integrated.

That is, the operation unit 118 functions as a GUI for the imaging staff to input the activation or stop (including the temporary stop) of the MRI apparatus 20, the selection of the pulse sequence, the imaging condition, the processing condition, and the like. The input of the imaging staff includes an input of designating only the sub coil channels necessary for imaging among the plurality of sub coil channels as described below.

As shown in FIG. 2, the above cameras 120A and 120B are disposed at both ends of the upper part of the bore 24. Each of the cameras 120A and 120B images the subject 100 and the like and outputs a video to the controller 116.

At least one of the camera 120A or 120B images a video including a face region of the subject 100. In addition, at least the other camera of the cameras 120A or 120B images the video including the receive coil unit 50.

The video captured by the cameras 120A and 120B is input to the controller 116 and is used as information for selecting only the sub coil channels necessary for imaging among a plurality of the sub coil channels as will be described below.

### Receive Coil Unit

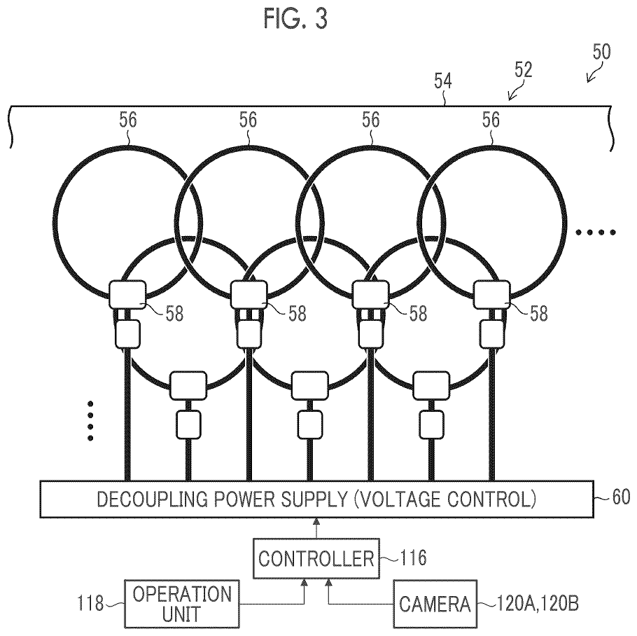

FIG. 3 is an explanatory diagram showing a configuration of main part of the receive coil 52 constituting the receive coil unit 50.

The receive coil 52 of the present example includes one sheet 54 that covers a wide imaging range as shown in FIG. 1. A plurality of sub coil channels 56 are disposed in the sheet 54.

The receive coil 52 has flexibility as a whole and is configured to be thin and light. By using the receive coil 52, it is possible to image various examination sites regardless of the physique of the subject 100. It should be noted that the receive coil 52 is an example of a receive coil according to the embodiment of the present invention.

As shown in FIG. 3, the receive coil 52 includes a plurality of sub coil channels 56. These sub coil channels 56 function as antennas that receive NMR signals.

The sub coil channel 56 is an annular coil having a diameter of about 10 cm to 15 cm as an example, and is two-dimensionally arranged inside the sheet 54 of the receive coil 52. The receive coil 52 of the present example is a multi channel coil and has 30 sub coil channels 56.

A decoupling circuit 58 is provided in each of the sub coil channels 56. Each of the decoupling circuits 58 is provided with a voltage-driven field effect transistor (FET). The FET of each decoupling circuit 58 is controlled ON/OFF by a driving voltage supplied from the decoupling power supply 60.

Each of the sub coil channels 56 is an antenna that receives the NMR signal generated from the biological tissue of the subject 100 and is adjusted to resonate at a specific frequency. The specific frequency is determined by the atomic nucleus (usually, a hydrogen atomic nucleus) to be observed in a biological tissue, and the magnetic field intensity.

Here, the transmitting coil 106 shown in FIG. 2 applies an RF pulse to the subject 100 to generate an NMR signal in an atomic nucleus of an atom constituting the biological tissue of the subject 100.

However, in a case where the sub coil channel 56 is brought into the resonance state (FET is ON) during the period in which the RF pulse is applied, the highly sensitive sub coil channel 56 receives an excessive RF magnetic field, and there is a risk that a peripheral circuit related to the sub coil channel 56 may be damaged.

Therefore, the decoupling power supply 60 switches the sub coil channel 56 to the detuned state (non-resonance state: FET is OFF) at least during a period in which the RF pulse is transmitted from the transmitting coil 106. In addition, the decoupling power supply 60 switches the sub coil channel 56 to a resonance state in order to receive the NMR signal generated from the biological tissue of the subject 100 after transmission of the RF pulse.

In addition, the decoupling power supply 60 switches, among the plurality of sub coil channels 56, only the sub coil channel 56 designated by the sub coil channel designation command, that is, the sub coil channel 56 necessary for imaging, to the resonance state, based on the sub coil channel designation command sent from the controller 116.

That is, the decoupling power supply 60 performs ON control of the FET of the decoupling circuit 58 of the sub coil channel 56 necessary for imaging and OFF control of the FET of the decoupling circuit 58 of the sub coil channel 56 unnecessary for imaging.

Therefore, in the receive coil 52 of the present example, only the sub coil channel 56 necessary for imaging is in the resonance state, and the sub coil channel 56 unnecessary for imaging is in the detuned state.

As a result, it is possible to solve a problem that an unnecessary signal is mixed from the sub coil channel 56 existing outside the field of view (FOV) and the image quality is deteriorated due to the wraparound artifact or the like. Although ON/OFF switching by voltage control of the FET is shown as an example in the decoupling circuit of FIG. 3, ON/OFF control can also be performed using switching by current control of a PIN diode or switching by voltage control of a micro electro mechanical systems (MEMS) device.

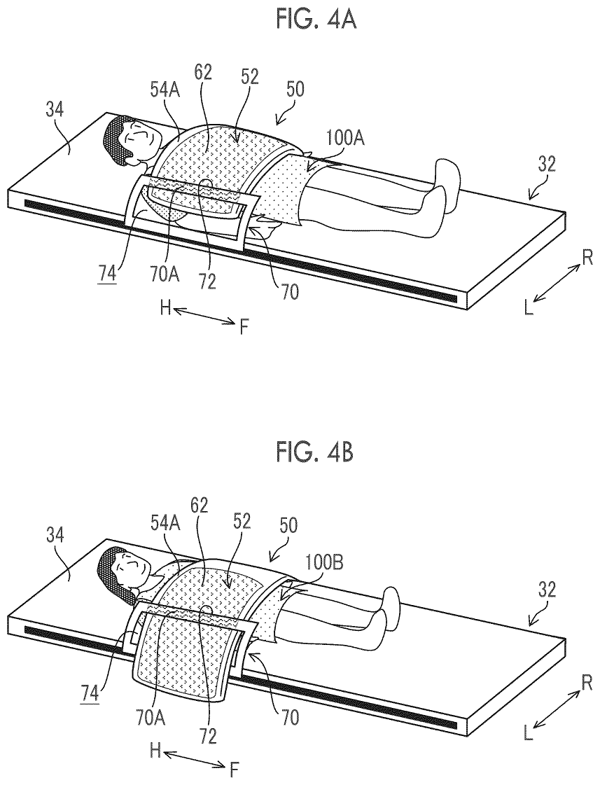

FIG. 4A is an explanatory diagram showing a state where a subject 100A having a large physique is fixed to the table 32 by the receive coil unit 50. In addition, FIG. 4B is an explanatory diagram showing a state where a subject 100B having a small physique is fixed to the table 32 by the receive coil unit 50.

As shown in FIGS. 4A and 4B, the receive coil unit 50 according to the present example is mounted on and used for the table 32 of the bed device 30. The receive coil unit 50 comprises a receive coil 52 and a fixing member 70. The fixing member 70 is an example of a fixing member according to the embodiment of the present invention.

One end side of the receive coil 52 is attached to a right side portion among both left and right side portions (LR) of the table 32. The receive coil 52 includes one sheet 54 that accommodates the plurality of sub coil channels 56 as described above. The sheet 54 is configured in a rectangular shape in plan view.

The member to be locked 62 is provided on a surface 54A of the sheet 54 (a surface opposite to the surface on the subject 100 side). The member to be locked 62 constitutes a member on one side of the surface fastener and is a member that is densely arranged and raised in a loop shape. The member to be locked 62 is provided in the entire area of the sheet 54 in the left and right (LR) direction except for the edge portion of the upper surface 54A of the sheet 54.

The fixing member 70 is attached to a left side portion of the table 32. The fixing member 70 includes a locking member 72 that is locked to the member to be locked 62. The fixing member 70 can fix the receive coil 52 to be length-adjustable in the left and right (LR) direction by adjusting a locking position of the member to be locked 62 with respect to the locking member 72.

The locking member 72 constitutes a member on the other side of the surface fastener and is a member that is raised in a hook shape. The locking member 72 and the member to be locked 62 can be attached to each other by being pressed against each other, and can be freely attached or detached.

The fixing member 70 of the present example is configured in a rectangular frame shape, and as shown in FIG. 4B, has an escape hole 74 for allowing the receive coil 52, which is an extra length portion after the length adjustment, to escape from the left side portion of the table 32 to the outside.

Here, the extra length portion of the receive coil 52 is inserted into the escape hole 74 from the back surface side (table 32 side) of the fixing member 70 and is escaped to the left side from the front surface side of the fixing member 70.

Therefore, the locking member 72 is provided on a back surface side of the upper side portion 70A of the fixing member 70 in order to lock the member to be locked 62. The upper side portion 70A is a side portion along the HF direction orthogonal to the LR direction (the length direction of the receive coil 52). Accordingly, the locking can be performed in a state where the extra length portion of the receive coil 52 is escaped from the escape hole 74, and the length of the receive coil 52 can be easily adjusted.

On the other hand, in a case where the fixing member comprising the escape hole 74 is not used, the extra length portion of the receive coil 52 may be folded toward the inner side of the table 32 in the subject 100B having a small physique.

In this folded portion, the sub coil channels 56 (including the ON-controlled sub coil channels 56 and the OFF-controlled sub coil channels 56) overlap with each other, which may affect the performance.

On the other hand, in the receive coil unit 50 of the embodiment, since the escape hole 74 is provided in the fixing member 70 and the extra length portion of the receive coil 52 is escaped to the outside of the table 32, the problem of the above-described overlapping can be solved, and the performance can be secured.

The member to be locked 62 and the locking member 72 are examples of a member to be locked and a locking member according to the embodiment of the present invention. In addition, the surface fastener is an example of the surface fastener according to the embodiment of the present invention. The surface fastener is known by a trade name such as a Magic Tape (registered trademark), a Velcro (registered trademark), and a Quicklon (registered trademark). In addition, the escape hole 74 is an example of an escape hole according to the embodiment of the present invention.

According to the receive coil unit 50 of the embodiment, one end side of one receive coil 52 is attached to the back side (right side portion) of the table 32 with the subject 100 interposed therebetween. The state before use of the receive coil 52 may be a state of being suspended on the right side of the table 32 or may be a state of being placed on the top plate 34. In the related art, it is common that the imaging staff carries the receive coil to the table in a case where the receive coil is placed in a receive coil placement and used. However, by permanently placing the receive coil on the table as described above, it is possible to reduce the work of carrying the receive coil 52 from the receive coil placement to the table 32. Therefore, according to the receive coil unit 50 of the embodiment, it is possible to reduce the burden on the imaging staff.

In addition, according to the receive coil 52 of the embodiment, the member to be locked 62 on one side of the surface fastener is provided on the receive coil 52, so that the receive coil 52 and the fixing belt are substantially integrated with each other. When used, the receive coil 52 is placed on the subject 100, and the other end side thereof is fixed to the fixing member 70 by using the surface fastener.

By adopting such a configuration, the receive coil 52 can be uniformly placed on the imaging part (for example, the abdomen) of the subject 100 regardless of the physique of the subject 100. Then, the subject 100 can be fixed to the table 32 only by locking the receive coil 52 and the fixing member 70 to each other using the surface fastener.

That is, in JP2019-92935A, the work of fixing the receive coil to the subject who is required to perform the two work steps can be performed by only one work step of locking the member to be locked 62 on the receive coil 52 side to the locking member 72 on the fixing member 70 side, and thus the number of work steps of the imaging staff can be reduced.

Therefore, according to the receive coil unit 50 of the embodiment, it is possible to reduce the burden on the imaging staff. In addition, since the length of the receive coil 52 can be adjusted by changing the locking position of the member to be locked 62 with respect to the locking member 72, the present invention can be applied regardless of the physique of the subjects 100A and 100B.

On the other hand, the MRI apparatus 20 according to the present example has a unit for automatically selecting the sub coil channel 56 necessary for imaging among the plurality of sub coil channels in accordance with the relative position of the fixing member 70 (locking member 72) and the receive coil 52 (member to be locked 62) in the left and right (LR) direction.

Accordingly, it is possible to reduce the efforts of selecting the sub coil channel 56 corresponding to the physique of the subject 100 and the imaging part among the plurality of sub coil channels 56. Therefore, it is possible to reduce the burden on the imaging staff.

Hereinafter, some automatic selection units (first and second automatic selection units) that automatically select the sub coil channel 56 necessary for imaging will be described.

### First Automatic Selection Unit

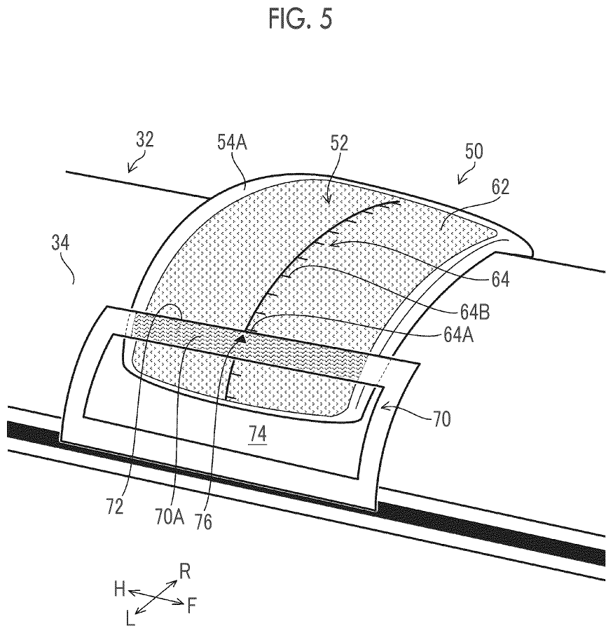

FIG. 5 is an explanatory diagram showing a main part of the receive coil unit 50 comprising the receive coil 52 provided with the gradations 64 and the fixing member 70 provided with the indicator 76.

As shown in FIG. 5, the receive coil 52 has gradations (number) 64 indicating the length of the sheet 54 in the left and right (LR) direction. The gradations 64 are provided on the surface 54A of the sheet 54.

The fixing member 70 includes an indicator 76. The indicator 76 is provided on the upper side portion 70A. This indicator 76 refers to a gradation corresponding to the locking position of the member to be locked 62 with respect to the locking member 72 in the gradations 64.

That is, the indicator 76 indicates a gradation corresponding to the physique of the subject 100. In FIG. 5, for convenience of description, the indicator 76 indicates the gradation 64A in the gradations 64.

In a case where the receive coil 52 is placed on a subject 100A having a large physique (see FIG. 4A), the indicator 76 indicates the gradation 64A (including the vicinity thereof). On the contrary, in a case where the receive coil 52 is placed on the subject 100B having a small physique (see FIG. 4B), the indicator 76 indicates the gradation 64B (including the vicinity thereof).

As described above, the physique of the subject 100 is identified by the gradation indicated by the indicator 76, and the sub coil channel 56 necessary for imaging is automatically selected from among the plurality of sub coil channels 56 based on the physique. That is, only the sub coil channel 56 necessary for imaging is ON-controlled, and the sub coil channel 56 unnecessary for imaging is OFF-controlled.

The indicator 76 indicates a gradation that is imaged by the camera 120A or the camera 120B shown in FIG. 2. The video captured by the camera 120A or the camera 120B is input to the controller 116 of FIG. 2 as a video signal.

The controller 116 selects the sub coil channel 56 required for imaging in accordance with a correspondence table between the input video (corresponding to the read gradation) and the sub coil channel 56 to be subjected to the ON control. The correspondence table is stored in advance in the ROM of the controller 116. Then, only the selected sub coil channel 56 is controlled to be ON during imaging. The camera 120A or the camera 120B is an example of a reading unit according to the embodiment of the present invention. In addition, the controller 116 is an example of a selection unit according to the embodiment of the present invention.

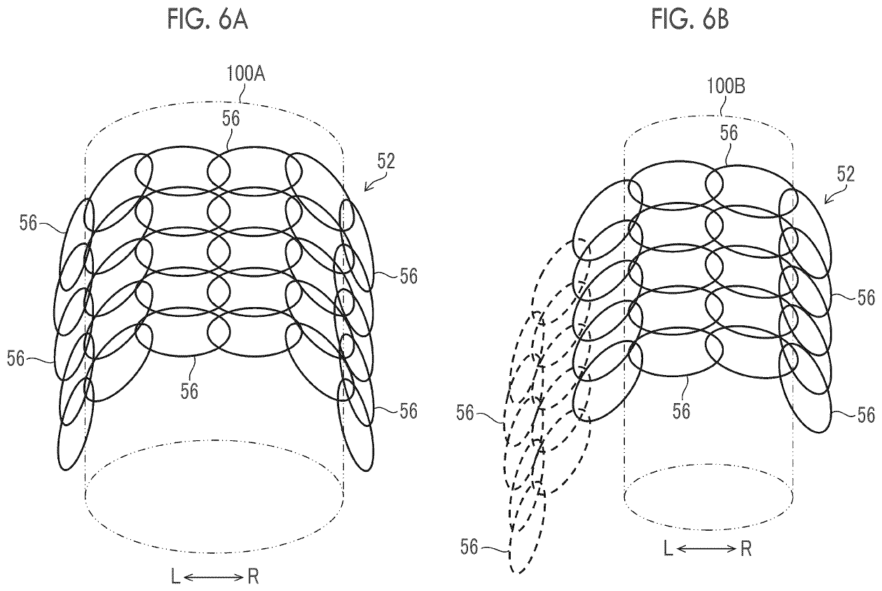

In FIG. 6A, in a case where the indicator 76 indicates the gradation 64A (including the vicinity thereof), the sub coil channel 56 selected by the controller 116 is indicated by a solid line. In this case, all (thirty) of the sub coil channels 56 are ON-controlled.

In FIG. 6B, in a case where the indicator 76 indicates the gradation 64B (including the vicinity thereof), the sub coil channel 56 selected by the controller 116 is indicated by a solid line, and the non-selected sub coil channel 56 is indicated by a broken line.

In this case, the twenty sub coil channels 56 disposed from the R side toward the L side are ON-controlled, and the ten sub coil channels 56 disposed on the L side are OFF-controlled. The ten sub coil channels 56 to OFF-controlled correspond to the sub coil channels 56 disposed in the extra length portion of the receive coil 52.

As described above, the first automatic selection unit can automatically select the sub coil channel 56 necessary for imaging from the video captured by the camera 120A or the camera 120B.

Accordingly, it is possible to reduce the labor of selecting the sub coil channels 56 corresponding to the physique of the subject 100 and the imaging part. As a result, it is possible to reduce the burden on the imaging staff.

In addition, it is possible to solve the problem that an unnecessary signal is mixed from the sub coil channel 56 existing outside the field of view (FOV) and the image quality is deteriorated due to the wraparound artifact or the like.

### Second Automatic Selection Unit

The imaging staff visually reads the gradation indicated by the indicator 76, and inputs the gradation (number) from a remote operation device (for example, a liquid crystal touch panel; not shown) comprising the gantry 22 or the operation unit 118.

The controller 116 selects the sub coil channel 56 necessary for imaging in accordance with a correspondence table between the gradation (number) thereof and the sub coil channels 56 to be ON-controlled. Then, only the selected sub coil channel 56 is controlled to be ON during imaging. The operation unit 118 is an example of an input unit according to the embodiment of the present invention. In addition, the controller 116 is an example of a selection unit according to the embodiment of the present invention.

The same effect as that of the first automatic selection unit can be obtained by the second automatic selection unit.

As in the first and second automatic selection units, by simplifying the degree of freedom of the relative position between the receive coil 52 and the fixing member 70 as one-dimensional information in the left and right direction, erroneous detection in a case where the position of the receive coil 52 is automatically detected can be suppressed. As a result, the selection accuracy of the sub coil channel 56 corresponding to the physique and the imaging part of the subject 100 is improved.

Hereinafter, some modification examples (first and second modification examples) according to the embodiment of the present invention will be described.

### First Modification Example

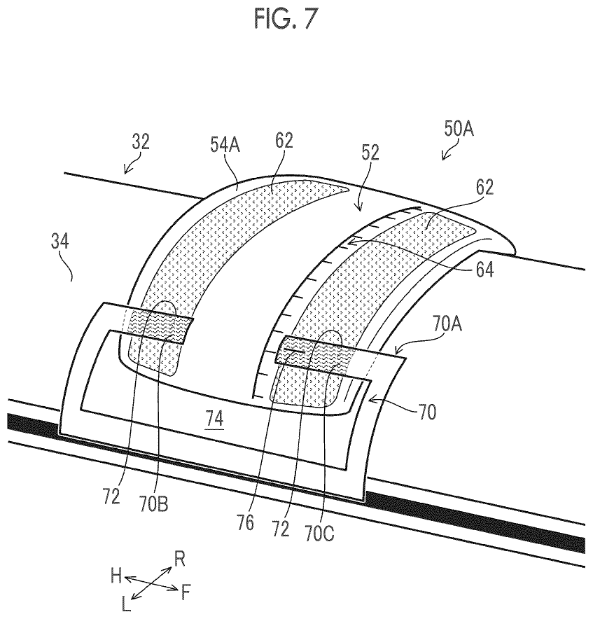

FIG. 7 is a perspective view of a main part of a receive coil unit 50A of the first modification example.

As shown in FIG. 7, in the fixing member 70 of the receive coil unit 50A, an upper side portion 70A of the fixing member 70 is divided into two parts at an interval in the HF direction.

The locking member 72 is provided on two back surface sides of the H side edge portion 70B and the F side edge portion 70C constituting the upper side portion 70A, respectively, and the locking member 72 locks the member to be locked 62 on the receive coil 52 side. Accordingly, the subject 100 can be fixed to the table 32 with sufficient strength.

In addition, since the H side edge portion 70B and the F side edge portion 70C are separated from each other at an interval in the HF direction, and the gradations 64 of the receive coil 52 are exposed in the space therebetween, the visibility of the gradations 64 is improved. The indicator 76 is provided in the H side edge portion of the F side edge portion 70C.

In addition, since the upper side portion 70A is separated into the H side edge portion 70B and the F side edge portion 70C, the area of the member to be locked 62 provided in the receive coil 52 can be reduced.

### Second Modification Example

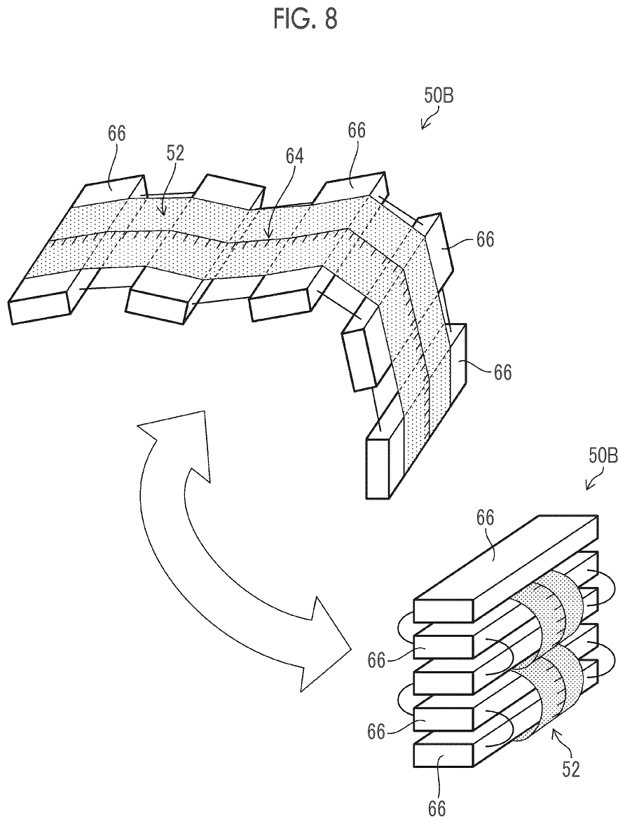

FIG. 8 is an explanatory diagram of a receive coil unit 50B of the second modification example. In FIG. 8, only the receive coil 52 is shown, and the fixing member 70 is not shown.

In the second modification example, a foldable receive coil is used as the receive coil 52. Specifically, in the receive coil 52, a plurality of plate-like bodies 66 that can be stacked are provided at intervals in a length direction of the receive coil 52. The plate-like body 66 is configured in a rectangular parallelepiped shape.

The receive coil 52 configured as shown in FIG. 8 can be folded like a folding screen. By folding the receive coil 52 like a folding screen, the storability is improved, and the receive coil 52 can be stored in a space-saving manner on the side of the table on the back side (R side).

In addition, by folding and storing the receive coil 52 in the vertical direction as shown in FIG. 8, the height of the table 32 in the vertical direction from the upper surface is higher than that in a case of a simple sheet-shaped receive coil. Therefore, it is easy to access the receive coil 52 from the front side (L side) of the table with the subject 100 interposed therebetween.

Since the receive coil 52 is in a fixed form using the surface fastener, the receive coil 52 in a folding screen shape is provided with the member to be locked 62 on a surface 54A (a surface opposite to the surface on the subject 100 side) in an open state. In FIG. 8, the member to be locked 62 is provided on the entire surface 54A of the receive coil 52, but the member to be locked 62 may be provided in a part of a region on the fixing member 70 (not shown) side.

As described above, the surface fastener comprises the locking member 72 on the convex surface (hook) side and the member to be locked 62 on the concave surface (loop) side. The surface of the folding screen-like receive coil 52 in the open state is frequently in contact with the skin of the imaging staff and the subject 100. Therefore, it is preferable that the member to be locked 62 having a small skin stimulus is provided on the surface of the receive coil 52.

### Third Modification Example

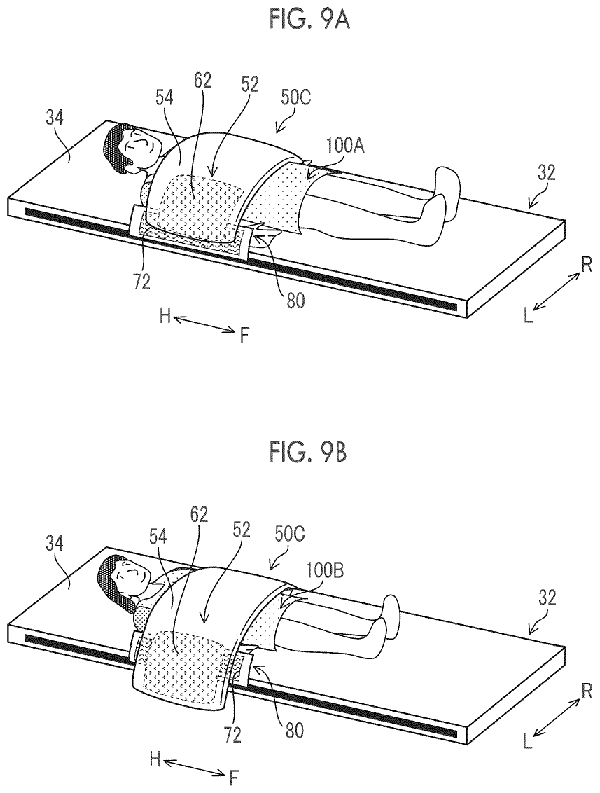

FIG. 9A is an explanatory diagram showing a state where a subject 100A having a large physique is fixed to the table 32 by the receive coil unit 50C of the third modification example. In addition, FIG. 9B is an explanatory diagram showing a state where the subject 100B having a small physique is fixed to the table 32 by the receive coil unit 50C.

Here, a difference in configuration between the receive coil unit 50 shown in FIGS. 4A and 4B and the receive coil unit 50C shown in FIGS. 9A and 9B will be described.

First, there is a point that the fixing member 70 of the receive coil unit 50 has the escape hole 74, whereas the fixing member 80 of the receive coil unit 50C does not have the escape hole 74.

The receive coil unit 50 is provided with the member to be locked 62 on the front surface side of the sheet 54 and is provided with the locking member 72 on the back surface side of the fixing member 70, whereas the receive coil unit 50C is provided with the member to be locked 62 on the back surface side of the sheet 54 and is provided with the locking member 72 on the front surface side of the fixing member 80.

The length of the fixing member 80 in the LR direction is shorter than the length of the fixing member 70 in the LR direction such that the fixing member 80 does not overlap with the subject 100A or 100B as much as possible.

With the receive coil unit 50C having such a configuration, after uniformly covering the imaging part of the subject 100A or 100B with the receive coil 52, the subject 100A or 100B can be fixed to the table 32 by only locking the receive coil 52 and the fixing member 80 to each other by using the surface fastener (the member to be locked 62 and the locking member 72).

In addition, even in a case where the fixing member 80 that does not have the escape hole 74 is used, by providing the member to be locked 62 on the back surface side of the sheet 54 and the locking member 72 on the front surface side of the fixing member 80, the extra length portion of the receive coil 50 can be escaped to the outside of the table 32. As a result, the problem of the overlap described above is solved, and the performance can be secured.

Since the receive coil unit 50 has a configuration in which the receive coil is escaped from the back surface side of the fixing member 70 and is passed through the escape hole 74, the fixing member 70 is unlikely to enter between the subject 100 and the receive coil unit 50. Therefore, there is an advantage in improving the adhesiveness of the receive coil 52 to the subject 100.

In the embodiment, the sub coil channel 56 necessary for imaging and the sub coil channel 56 unnecessary for imaging are selected in advance, and the ON/OFF control of each sub coil channel 56 is performed for imaging, but the present invention is not limited to this.

For example, all the sub coil channels 56 may be set to ON at the time of imaging, and only the data to be used may be selected from all the data obtained from all the sub coil channels 56 at the time of image generation to generate an image.

In addition, in the present example, an example in which the surface fastener is used as the length adjustment unit of the receive coil 52 has been described, but the present invention is not limited thereto. That is, a fixing unit other than the surface fastener may be used as long as the unit can adjust the length in the left and right direction in accordance with the physique of the subject 100.

For example, a non-metallic slide belt type receive coil unit including a buckle (fixing member) capable of adjusting and fixing the length of the receive coil 52 in a stepless manner may be used. Even in this receive coil unit, the length in the left and right direction can be adjusted according to the physique of the subject 100.

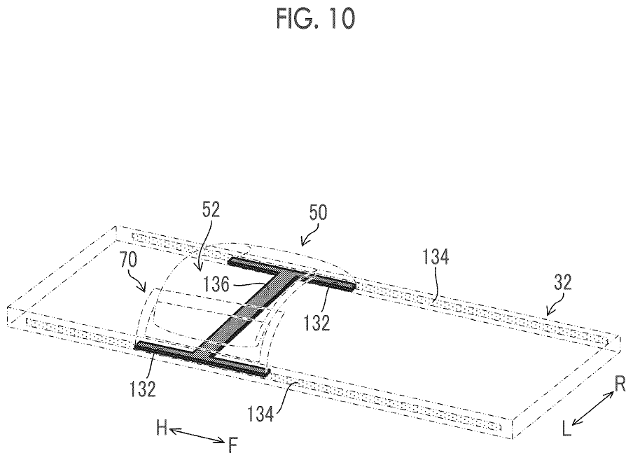

FIG. 10 shows a form in which the receive coil unit 50 is provided to be slidable in the HF direction with respect to the table 32.

According to FIG. 10, a slider 132 is provided in each of the fixing member 70 and the receive coil 52, and each of the sliders 132 is slidably connected to rails 134 and 134 in the HF direction provided on the left and right side portions of the table 32.

Therefore, by sliding the fixing member 70 and the receive coil 52 along the rails 134 and 134 to change the positions, it is possible to easily perform the position adjustment in the HF direction required in accordance with the difference in physique of the subject 100 or the change in the imaging part.

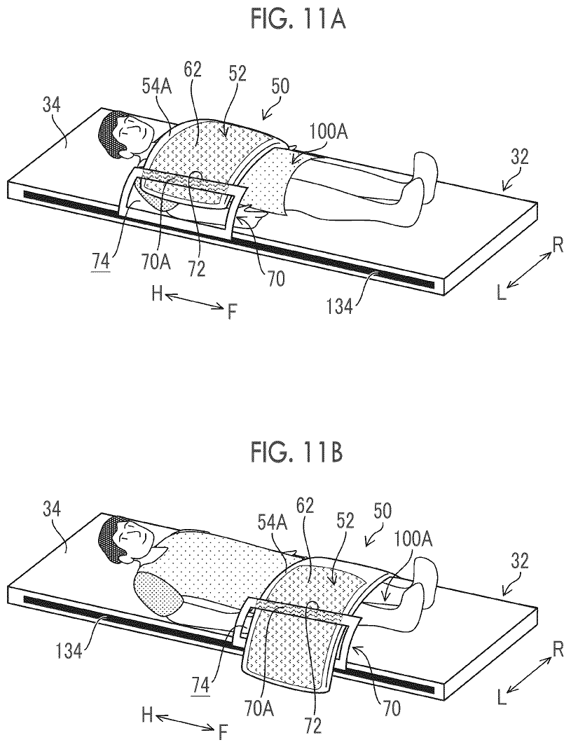

Here, FIG. 11A shows a form in which the receive coil 50 is positioned on the chest of the subject 100, and FIG. 11B shows a form in which the receive coil 50 is positioned on the leg part of the subject 100. As described above, by providing the fixing member 70 and the receive coil 52 so as to be slidable in the HF direction, it is easy to adjust the position in the HF direction in accordance with the change of the imaging part.

Further, as shown in FIG. 10, by providing a connecting member 136 that connects the fixing member 70 and the receive coil 52 inside the table 32, the positions of the fixing member 70 and the receive coil 52 in the HF direction can be moved simultaneously. That is, the imaging staff remains standing on the front side (fixing member 70 side) of the table 32, and thus it is easy to adjust the position of the receive coil 52 on the back side of the table by only moving the fixing member 70.

Although the embodiments of the receive coil unit and the medical image diagnosis system according to the present invention have been described above, the present invention is not limited to the above-described embodiments, and various improvements or modifications may be made without departing from the scope of the present invention.

### Explanation of References

10: medical image diagnosis system
20: MRI apparatus
30: bed device
32: table
34: top plate
50: receive coil unit
50A: receive coil unit
50B: receive coil unit
50C: receive coil unit
52: receive coil
54: sheet
56: sub coil channel
62: member to be locked
64: gradations
70: fixing member
72: locking member
74: escape hole
76: indicator
80: fixing member
116: controller
118: operation unit
120A: camera
120B: camera

What is claimed is:

1. A receive coil unit comprising:
a receive coil of which one end side of a table, on which a subject is placed, is attached to one side portion among both side portions of the table, that has one sheet accommodating a plurality of sub coil channels, and that has a member to be locked provided on a surface of the sheet; and
a fixing member that is attached to the other side portion opposite to the one side portion of the table, that has a locking member locked to the member to be locked, and that fixes the receive coil to be length-adjustable by adjusting a locking position of the member to be locked with respect to the locking member.

2. The receive coil unit according to claim 1,
wherein the member to be locked and the locking member are surface fasteners.

3. The receive coil unit according to claim 2,
wherein the member to be locked is provided on a surface opposite to a surface on a subject side among two front and back surfaces of the sheet.

4. The receive coil unit according to claim 1,
wherein the fixing member has an escape hole for leading an extra length portion of the receive coil after length adjustment to an outside of the table.

5. The receive coil unit according to claim 1,
wherein the receive coil is foldable.

6. The receive coil unit according to claim 1,
wherein the receive coil has gradations indicating a length of the sheet, and
the fixing member has an indicator indicating a gradation corresponding to the locking position of the member to be locked with respect to the locking member among the gradations.

7. A medical image diagnosis system comprising:
the receive coil unit according to claim 1; and
a magnetic resonance imaging apparatus.

8. The medical image diagnosis system according to claim 7,
wherein the receive coil has gradations indicating a length of the sheet,
the fixing member has an indicator indicating a gradation corresponding to the locking position of the member to be locked with respect to the locking member among the gradations, and
the magnetic resonance imaging apparatus includes
a reading unit configured to read the gradation indicated by the indicator, and
a selection unit configured to select a sub coil channel to be driven among the plurality of sub coil channels corresponding to the gradation read by the reading unit.

9. The medical image diagnosis system according to claim 7,
wherein the receive coil has gradations indicating a length of the sheet,
the fixing member has an indicator indicating a gradation corresponding to the locking position of the member to be locked with respect to the locking member among the gradations, and
the magnetic resonance imaging apparatus includes
an input unit configured to input the gradation indicated by the indicator, and
a selection unit configured to select a sub coil channel to be driven among the plurality of sub coil channels corresponding to the gradation input by the input unit.
